(19) United States
(12) Patent Application Publication
(10) Pub. No.: US 2022/0256879 A1
(43) Pub. Date: Aug. 18, 2022

(54) CRYO-CRYSTALLISED FAT

(71) Applicant: Arthur Branwell & Co Limited, Essex (GB)

(72) Inventors: William Henry DENYER, Essex (GB); Cheryl LOCKETT, Essex (GB); Kenneth LAIDLAW, Essex (GB)

(21) Appl. No.: 17/627,896

(22) PCT Filed: Jul. 20, 2020
(86) PCT No.: PCT/GB2020/000065

§ 371 Date: Jan. 18, 2022

(30) Foreign Application Priority Data
Jul. 18, 2019 (GB) 1910271.4

(51) Int. Cl. A23D 9/05; C07C 69/30

(52) U.S. Cl. CPC A23D 9/05; A21D 2/165; C07B 2200/13; C07C 69/30

(57) ABSTRACT

A cryo-crystallised particulate fat and method for producing such a fat, having reduced D[4,3], D[3,2] and D(50) than particulate fats known in the art, as measured by laser diffraction. Particles are formed by alterations to product throughput, air pressure and flow rate. The particles offer increased functionality in various food applications.

### BACKGROUND OF THE INVENTION

The invention provides fat having enhanced functionality, a process for producing it cryogenically, and products containing or derived from it.

As used herein “fat” means a triglyceride of one or more fatty acids which is solid under ambient storage conditions.

“Fatty acid” shall mean a synthetic or preferably natural branched or preferably straight chain alkenyl, hydroxyalkyl or preferably alkyl carboxylic acid having from 10 to 25 carbon atoms.

Cryogenically processed fat crystals are sometimes used in the food industry, for example as a structurant in spreads and shortenings and as an adjuvant to bakery products, non-meat protein substitutes and animal feeds. They are conventionally produced by spraying molten fat into a cryogenic chamber in which the fat droplets are rapidly cooled by contact with jets of liquefied gas such as nitrogen or carbon dioxide to form fine crystals, which exhibit substantial performance advantages compared to non-cryogenically formed fat particles. These advantages include improved stability of emulsions and oleogels and improved functionality, resulting in a reduction in the amount of fat required in the final food product.

The cryogenic treatment of fat has been described for example in EP 0393963, EP 1238589 and U.S. Pat. No. 9,011,951.

There is a demand for food products with reduced fat content. There is also a demand for products with fewer extraneous additives such as emulsifiers, preservatives, stabilisers, flavourings and colourants not found in traditionally prepared food but hitherto deemed essential to provide manufacturing tolerance, product consistency, shelf life and an acceptable appearance and taste for industrial manufactured food products. This is reflected in a move toward “clean” labelling, i.e. the removal of ingredients whose presence is required to be identified on the label by code numbers or chemical names whose significance the average consumer does not understand.

We have now discovered a method of forming a cryogenically treated fat with a novel composition conferring enhanced functionality. The novel product possesses a greatly reduced particle size and is rapidly dispersed into bakery doughs, batters or pastes, enabling the reduction or elimination of selected powdered fats or emulsifiers. It also readily forms stable emulsions and oleogels reducing or eliminating the need for extraneous emulsifiers, and delivers enhanced performance in a variety of applications including bakery shortenings and animal feedstuffs.

Conventional cryo crystallised fat has a mean particle size (measured by laser diffraction and expressed as D[3,2] or surface mean diameter) of about 100 microns. It has been reported in three polymorphic forms, namely the metastable alpha, the beta′, which has been identified as the most effective functionally and the thermally stable beta. On long term storage the alpha transforms into the beta′, which in term transforms into the beta.

We have discovered that when the rate of cooling in a cryogenic crystalliser is increased substantially, compared to that currently employed, e.g. by increasing the ratio between the rate of supply of the coolant and that of the molten fat feed, a product is obtained with significantly reduced particle size (e.g D[3,2] less than 60, typically less than 40). In certain cases, a previously unreported amorphous form and an increased total proportion of amorphous and alpha relative to beta′ and beta in the freshly prepared product has also been observed.

### SUMMARY OF THE INVENTION

According to a first embodiment, a particulate fat is provided, having either a D[3,2] between 10 and 80 microns, or a D[4,3] between 20 and 160 microns or a D(50) between 20 and 160 microns.

Preferably, having a D[3,2] between 10 and 50 microns, a D[4,3] between 30 and 140 microns and a D(50) between 30 and 140 microns.

More preferably, having a D[3,2] between 10 and 40 microns, a D[4,3] between 40 and 120 microns and a D(50) between 40 and 120 microns.

More preferably, having a D[3,2] between 20 and 40 microns, a D[4,3] between 50 and 100 microns and a D(50) between 50 and 100 microns.

Most preferably, having a D[3,2] between 25 and 35 microns, a D[4,3] between 70 and 90 microns and a D(50) between 60 and 80 microns.

Advantageously, having a major proportion of crystalline material.

Preferably, comprising α, β and β′ poly-morphs and optionally, amorphous fat.

Preferably, the crystalline portion comprises an α content between 5 and 55%, a β content between 40 and 70% and a β′ content between 15 and 33% by weight.

More preferably, the crystalline portion comprises an α content between 7 and 50%, a β content between 40 and 65% and a β′ content between 20 and 32% by weight.

Most preferably, the crystalline portion comprises an α content between 8 and 45%, a β content between 40 and 60% and a β′ content between 21 and 31% by weight.

According to a second embodiment, the invention provides a method of preparing a particulate fat which comprises forming said fat into molten droplets; forcing said fat under pressure through an atomising nozzle into a cryogenic chamber; cooling said droplets at a rate between 1000° C./s and 2000° C./s to form particles of at least partially crystallised fat, having either a D[3,2] between 10 and 80 microns, or a D[4,3] between 20 and 160 microns, or a D(50) between 20 and 160 microns.

Preferably, the rate of cooling is between 1200° C./s and 1800° C./s.

More preferably, the rate of cooling is between 1400° C./s and 1700° C./s.

Most preferably, the rate of cooling is between 1600° C./s and 1700° C./s.

Advantageously, the air flow rate through the atomising nozzle is between 1000 and 3000 litres/minute.

More advantageously, the air flow rate through the atomising nozzle is between 2000 and 3000 litres/minute.

More advantageously, the air flow rate through the atomising nozzle is between 2300 and 3000 litres/minute.

Most advantageously, the flow rate through the atomising nozzle is between 2500 and 3000 litres/minute.

Preferably, the product throughput is between 100 and 900 kg/hour.

More preferably, the product throughput is between 250 and 500 kg/hour.

Most preferably, the product throughput is between 300 and 400 kg/hour.

Advantageously, the pressure across the atomizing nozzle is between 2 and 8 bar.

More advantageously, the pressure across the atomizing nozzle is between 4 and 7 bar.

Most advantageously, the pressure across the atomizing nozzle is between 5 and 6 bar. Preferably, said fat comprises a major proportion of crystalline material.

According to a further embodiment the invention provides a method of preparing a particulate fat which comprises forming said fat into molten droplets and cooling said droplets at a sufficient rate to form particles of at least partially crystallised fat having a D[3,2] less than 60 microns, preferably less than 50 microns, more preferably less than 40 microns most preferably less than 35 microns, as determined by laser diffraction.

According to a further embodiment, the invention provides a suspension of fat particles as described in any one of the above embodiments in oil.

According to a further embodiment, the invention provides a bakery mix comprising said fat particles.

According to a further embodiment, the invention provides a spread comprising particles of fat as described in any one of the above embodiments.

According to a further embodiment, the invention provides an animal feedstuff containing fat particles as described in any one of the above embodiments.

According to a further embodiment, the invention provides a consumer product containing fat particles as described in any one of the above embodiments and substantially free from extraneous emulsifiers or stabilisers.

### DETAILED DESCRIPTION OF THE EMBODIMENTS

As used herein “substantially free” means present in amounts insufficient to require separate itemisation on product labelling.

As used herein, D[4,3] relates to the volume mean of a sample as measured by laser diffraction. D[3,2] relates to the surface mean of a sample as measured by laser diffraction. D(50) relates to the population median of a sample as measured by laser diffraction. As used herein in relation to the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The fat may comprise glycerides of octanoates, decanoates, dodecanoates, laurates, myristates, stearates, iso stearates, oleates, linoleates, linolenates, ricinoleates, behenates, erucates, palmitates, eicosapentaenoates, docosahexaenoates and mixtures thereof, and especially mixtures derived from the saponification of rape oil, coconut oil, palm oil, olive oil, sunflower oil, corn oil or other vegetable oils, tree nut oil, tallow and/or other animal fats. Preferred are fats derived from palm or fully hydrogenated vegetable oils.

The fat particles preferably have D[3,2] greater than one, more preferably greater than 10, still more preferably greater than 20, most preferably greater than 25 microns.

The cryo crystallised fat is preferably prepared by heating the feed to a temperature above its melting point but below its decomposition temperature and forcing it under pressure through an appropriately sized nozzle into a cryogenic chamber, in which it is contacted with a liquefied gas such as liquid air or nitrogen, injected into the chamber under pressure. The temperature and nozzle diameter are selected to provide a fine spray. The temperature is preferably the minimum consistent with a free-flowing feed.

The rate of cooling, using an air atomising nozzle, may be controlled by varying the air pressure. The higher the air pressure and the lower the flow of feed, the greater the rate of cooling.

The rate of cooling is preferably greater than 1000° C. per second, more preferably greater than 1200° C. per second, still more preferably greater than 1400° C. per second, yet more preferably greater than 1500° C. per second, most preferably greater than 1600° C. per second.

The novel cryo crystallised fat may be used in breadmaking to replace the emulsifiers which are commonly required in industrial bakery to obtain an acceptable product. The most commonly used include sodium stearoyl lactylate (SSL) and the diacetyl ester of mono/di glycerides (DATEM). These two emulsifiers can be used in combination in order to obtain optimum effects.

The novel fat may typically be added to dough at the mixing stage in proportions of at least 0.05%, preferably over 0.07%, most preferably greater than 0.09% based on the weight of dough. It is generally unnecessary to use more than 0.2%, preferably less than 0.15%, most preferably less than 0.12% of the fat expressed as a % of flour. The latter can be used in equivalent proportions to the conventional emulsifiers without changing the recipe and without any reduction in quality of the product.

Typically, the dough will contain flour in a proportion of at least 50% preferably at least 55% most preferably more than 57% by weight, based on the total weight of the dough but less than 70%, preferably less than 65%, most preferably less than 62%. The dough may typically contain at least 30%, preferably at least 35%, and up to 45% but preferably less than 40% by weight of water based on the weight of the dough.

The novel fat can be used as a substitute for mixtures of hard fat and/or emulsifier in formulations employing a compound dough conditioner. The novel conditioner typically contains the cryo crystallised fat in similar proportions to the hard fat/emulsifier compound in conventional conditioners, e.g. at least 10%, preferably more than 15%, most preferably more than 18%, and up to 30% but preferably less than 25%, most preferably less than 22% based on the total weight of conditioner.

The balance of the conditioner may comprise flour or starch as a carrier, typically in proportions of greater than 30%, preferably greater than 40%, most preferably greater than 45%, and up to 75%, preferably less than 60%, most preferably less than 65% by weight based on the total weight. The conditioner may contain other ingredients commonly included in such conditioners such as gluten, e.g. gluten, typically in proportions from 20 to 30%.

The novel fat can be used to make shortenings comprising a vegetable oil such as palm, rapeseed, sunflower, olive or corn oil. Typically, the shortenings contain more than 50%, preferably more than 60%, even more preferably more than 70% oil and more than 5% preferably more than 10%, more preferably more than 15%, most preferably more than 20% fat. The suspensions exhibit improved stability compared to conventional oil/fat shortenings and are preferably substantially free from emulsifiers that would require separate listing on the label.

The invention also comprises mixtures of the novel fat with minor proportions of carriers such as flour and effective amounts of dough enhancers such as enzymes, for use as additives to dough.

The novel fat may be used as a replacement for hard fat flakes commonly used as a structurant in formulations such as pastry, biscuits and pizza crust, giving improved dispersion, a finer crumb structure and a reduction in total fat levels by up to about 50%.

In processes wherein dough balls are dusted with flour prior to resting, including yeasted doughnuts and flat breads such as tortilla and pizza, the flour may be replaced by our novel fat to reduce the amount of allergenic dust in the bakery atmosphere and the frequency with which equipment requires cleaning, as well as reducing the frequency with which any frying oil needs replacing and providing a brighter baked or fried surface.

In such dusting applications the novel fat may applied indirectly, e.g. to prooving belts and trays, or directly to the doughballs, e.g, using vibratory or rotary sprinklers.

The novel fat may be used in sugar-based formulations such as fondants, icings and fillings containing more than 60%, preferably more than 70%, more preferably more than 75% of sugar. The sugar typically comprises sucrose and/or glucose. The fat is typically present in proportions of at least 3, more preferably greater than 4, most preferably greater than 5%, but less than 12, more preferably less than 10, most preferably less than 8%, based on the weight of the composition. Other ingredients may comprise water, vegetable oil, glycerine, flavourings, colourants, preservatives and/or aroma.

The sugar-based formulations of our invention have improved (smoother and more luxuriant) mouth feel and offer the chance to reduce fat levels.

The fat of the present invention may be applied after baking to the hot products at a temperature sufficient enough to melt the fat and form, on cooling, a protective glaze inhibiting the passage of moisture, or for basting meat, fish and vegetables on barbecues, rotisseries, griddles and the like.

Other potential uses for the present novel fat include as a carrier and/or dispersant for active ingredients in culinary, cosmetic and/or pharmaceutical applications, and as an organoleptically enhancing component of dairy desserts and beverages such as milk shakes and smoothies and of non-dairy creams.

### EXAMPLES

The invention will be illustrated by the following examples in which all proportions are by weight based on the total weight of the formulation unless stated to the contrary.

### Example 1

Palm stearin was sprayed into a cryo crystalliser and rapidly chilled with liquid nitrogen, using the settings described in Table I, which provided a calculated rate of cooling of 1620° C. per second. The control was a standard commercial product cooled at a calculated rate of 740° C. per second. The total time taken for cooling to the end of fusion temperature at 44.1° C. was 93 milliseconds for the method known in the art, compared to 40 milliseconds for the novel method reported herein. The example had D[3,2] of 33.4 microns measured by laser diffraction, compared with 99.1 for the control; a D[4,3] of 87.6 microns, compared with 201 for the control; and D(50) of 73.9 microns, compared with 190 for the control. The average air flow rate utilised for the novel process was 2851 litres/minute, whereas the average flow rate utilised in the control (exemplifying processes known in the art) was 736 litres/minute.

TABLE I

| Parameter | Example 1 | | Control | |
|---|---|---|---|---|
| Temperature of fat prior to process | 70-75° | C. | 70-71° | C. |
| Paddle mixing speed (prior to Cryo- | Intermittent/slow | | Intermittent/slow | |
| Crystalliser) | | | | |
| Air Flow rate to atomising nozzle | 2830-2870 | | 720-760 | |
| (litres/min)) | | | | |
| Product (liquid) pressure onto nozzle (bar) | 2.7 | | 1.2 | |
| Air pressure into nozzle (bar) | 5.6 | | 1.4 | |
| Nitrogen temperature delivered to Cryo | 185° | C. | 185° | C. |
| Chamber | | | | |
| Nitrogen use ratio to product (%) | 68-71 | | 56-59 | |
| Nitrogen throughput (kg/Hr) | 260-2.80 | | 590-610 | |
| Product throughput (kg/Hr) | 360-380 | | 990-102.0 | |
| Storage Temperature (after production) | 10° | C. | 10° | C. |

| | | |
|---|---|---|
| Storage (Maturation) period | 3 days minimum | 3 days minimum |

The resulting novel cryo-crystallised fat presented with the following polymorphic ratios (as percentage weight); 18.7% α, 52.0% β and 29.4% β′. By comparison, the fat produced via the control method noted in Example 1, representing fats known in the art and produced via conventional methods, presented with polymorphic ratios as follows; 28.3% α, 44.9% β and 26.8% β′. The novel process is believed to stabilise the α form of the novel fat particles and rapid cooling leads to increased α content.

Scanning Electron Microscopy analysis of the novel fat in comparison to that of the control (prior art) showed that the novel fat particles were smaller, tear shaped and presented with greater dispersion; whereas the control particles were typically larger, more polydisperse (with some smaller and spherical, whilst others were irregularly shaped and larger) and presented with greater levels of particle agglomeration. The novel fat particles have textured and irregular surfaces, whilst the majority of the control particles present with smooth and more-uniform surfaces.

The size, shape and increased number of novel fat particles relative to those produced in the conventional (known) method subsequently provides a greater surface area per volume of fat, increasing the interface with air bubbles during baking and leading to improvements in desirable product metrics.

### Example 2

Example 1 was repeated using fully saturated rapeseed in place of palm. The D[3,2] of the product was 28.7 microns, the D[4,3] was 75.0 microns and the D(50) was 61.63 microns.

### Example 3

The products of Examples 1 and 2 were each added to sunflower oil in a proportion of 1:4 by weight and blended in a high shear mixer for 3 minutes at 2500 rpm followed by 1 minute at 5000 rpm. In each case the product was a stable, pumpable shortening, free from emulsifiers, which provided a softer crumb than the currently preferred commercial shortenings and conferred improved product quality and extended organoleptic shelf life when use in both bread and cake trials.

### Example 4

A test bake compared the average volume of loaves baked using DATEM and SSL emulsifiers alone and in combination, a standard cryo crystallised fat alone, and each of the products of Examples 1 and 2 alone. The results are set out in Table II.

TABLE II

| Average Volume | mls |
|---|---|
| Control (blank) | 2646.73 |
| SSL | 2696.81 |
| DATEM | 2786.37 |
| SSL + DATEM | 2927.77 |
| Standard Cryo fat | 2845.19 |
| Example 1 | 2934.74 |
| Example 2 | 3041.36 |

It will be seen that neither SSL nor DATEM alone gave a significant improvement in baked volume. These two emulsifiers are commonly used in conjunction and must be separately identified by their E numbers on all labelling.

The standard cryo crystallised fat did not provide an adequate alternative, but the products of the invention delivered improved dough handling (as a drier dough) and tolerance combined with greater baked volume and enhanced crumb structure compared to all the emulsifiers tested.

### Example 5

A combination of SSL, DATEM and a commercial shortening comprising a suspension of palm fat in oil sold under the name “AMBREX BREAD FAT SG” was compared with a combination of the product of example 2 and the shortening of example 3. The former gave an average baked volume of 3116.1 cc; The latter gave an average baked volume of 3185 cc. In addition, the use of example 2 combined with example 3 delivered a softer crumb when measured by texture analysis compression test throughout shelf life.

### Example 6

Fat according to example 2 is added to the mixing howl in a standard bread making recipe as a direct replacement for the crumb strengthening emulsifiers. No other change in the recipe is required, as shown in Table III

TABLE III

| Ingredient | Standard | New |
|---|---|---|
| Wheatflour | 60.88 | 60.88 |
| Water | 36.52 | 36.52 |
| Yeast | 1.52 | 1.52 |
| Salt | 0.97 | 0.97 |
| DATEM | 0.045 | 0 |
| SSL | 0.045 | 0 |
| Example 2 | 0 | 0.9 |
| Enzyme | qs | qs |
| Ascorbic Acid | qs | qs |

### Example 7

A compound conditioner comprising hard fat and emulsifier in a farinaceous or starch-based carrier is often supplied for direct addition to the mixing bowl. Fat according to the invention may be substituted for the hard fat and emulsifier, as shown in Table IV

TABLE IV

| Component | Standard | New |
|---|---|---|
| Wheatflour or Starch | 49.26 | 49.26 |
| carrier | | |
| Hard Fat (>40° C. MP) | 10 | 0 |
| DATEM E472e | 10 | 0 |
| Vital Wheat Gluten | 25 | 25 |
| Example 1 | 0 | 20 |
| Enzyme(s) | 4.24 | 4.24 |
| Ascorbic Acid | 1.5 | 1.5 |

### Example 8

Hard fat particles are commonly included in pastry recipes, e.g. for pizzas and biscuits. Replacement with fat of the invention provides an improved dispersion and crumb structure and reduced fat, as in the pizza recipe in Table V, in which all percentages are by weight based on the weight of flour.

TABLE V

| Ingredient | Standard | New |
|---|---|---|
| Bread Flour | 100 | 100 |
| Water | 50 | 50 |
| Yeast | 3.5 | 3.5 |
| Salt | 1.75 | 1.75 |
| Sugar | 2 | 2 |
| Vegetable oil | 5 | 4 |
| Hard fat crystals | 8 | 0 |
| Example 2 | 0 | 5 |
| Leavening | 1.5 | 1.5 |
| Enzyme | 0.1 | 0.1 |

### Example 9

The ingredients for a fondant using the product of example 1 are set out in Table VI.

TABLE VI

| Ingredient | % |
|---|---|
| Celebration sugar | 79.15 |
| Example 1 | 6.33 |
| Water at 90° C. | 5.94 |
| Vegetable oil | 3.96 |
| Glycerine | 3.96 |
| Liquid flavour | 0.32 |
| Emulsifier | 0.16 |
| Potassium sorbate | 0.16 |
| Liquid colouring | 0.04 |

Using a jacketed mixing bowl at 40° C. blend the sugar with hot water to form a fondant, then add the remaining ingredients and mix thoroughly.

### Example 10

The ingredients of a tortilla dough containing the product of Example 2 are set out in Table VII, expressed as percentage by weight based on the weight of flour

TABLE VII

| Ingredient | Standard | New |
|---|---|---|
| Wheat flour | 100 | 100 |
| Water | 50 | 50 |
| Palm shortening | 10 | 0 |
| Vegetable oil | 0 | 3 |
| Example 2 | 0 | 7 |
| Leavening | 1.8 | 1.8 |
| Salt | 1.2 | 1.2 |
| Conditioner | 1 | 1 |
| Sugar | 1 | 1 |
| Emulsifier | 0.75 | 0.75 |
| Calcium propionate | 0.5 | 0.5 |
| Xanthan gum | 0.3 | 0.3 |
| Guar gum | 0.3 | 0.3 |
| Sorbic acid | 0.3 | 0.3 |
| Fumaric acid | 0.3 | 0.3 |
| Carboxymethyl | 0.1 | 0.1 |
| cellulose | | |

The invention provides a brighter crumb and surface, with reduced fat levels.

1. A particulate fat, having;
either a D[3,2] between 10 and 80 microns, or a D[4,3] between 20 and 160 microns or a D(50) between 20 and 160 microns.

2. The particulate fat as claimed in claim 1, having a D[3,2] between 10 and 50 microns, a D[4,3] between 30 and 140 microns and a D(50) between 30 and 140 microns.

3. The particulate fat as claimed in claim 1, having a D[3,2] between 10 and 40 microns, a D[4,3] between 40 and 120 microns and a D(50) between 40 and 120 microns.

4. The particulate fat as claimed in claim 1, having a D[3,2] between 20 and 40 microns, a D[4,3] between 50 and 100 microns and a D(50) between 50 and 100 microns.

5. The particulate fat as claimed in claim 1, having a D[3,2] between 25 and 35 microns, a D[4,3] between 70 and 90 microns and a D(50) between 60 and 80 microns.

6. The particulate fat of claim 1, having a major proportion of crystalline material.

7. The particulate fat as claimed in claim 1, comprising α, β and β′ poly-morphs and optionally, amorphous fat.

8. The particulate fat as claimed in claim 7, wherein the crystalline portion comprises an α content between 5 and 55%, a β content between 40 and 70% and a β′ content between 15 and 33% by weight.

9. The particulate fat as claimed in claim 8, wherein the crystalline portion comprises an α content between 7 and 50%, a β content between 40 and 65% and a β′ content between 20 and 32% by weight.

10. The particulate fat as claimed in claim 9, wherein the crystalline portion comprises an α content between 8 and 45%, a β content between 40 and 60% and a β′ content between 21 and 31% by weight.

11. A method for preparing a particulate fat, comprising;
forming said fat into molten droplets;
forcing said fat under pressure through an atomising nozzle into a cryogenic chamber;
cooling said droplets at a rate between 1000° C./s and 2000° C./s to form particles of at least partially crystallised fat, having either a D[3,2] between 10 and 80 microns, or a D[4,3] between 20 and 160 microns, or a D(50) between 20 and 160 microns.

12. The method of preparing a particulate fat as claimed in claim 11, wherein the rate of cooling is between 1200° C./s and 1800° C./s.

13. The method of preparing a particulate fat as claimed in claim 12, wherein the rate of cooling is between 1400° C./s and 1700° C./s.

14. The method of preparing a particulate fat as claimed in claim 13, wherein the rate of cooling is between 1600° C./s and 1700° C./s.

15. The method of preparing a particulate fat as claimed in claim 11, wherein the flow rate through the atomising nozzle is between 1000 and 3000 litres/minute.

16. The method of preparing a particulate fat as claimed in claim 15, wherein the flow rate through the atomising nozzle is between 2000 and 3000 litres/minute.

17. The method of preparing a particulate fat as claimed in claim 16, wherein the flow rate through the atomising nozzle is between 2300 and 3000 litres/minute.

18. The method of preparing a particulate fat as claimed in claim 17, wherein the flow rate through the atomising nozzle is between 2500 and 3000 litres/minute.

19. The method of preparing a particulate fat as claimed in claim 11, wherein the product throughput is between 100 and 900 kg/hour.

20. The method of preparing a particulate fat as claimed in claim 19, wherein the product throughput is between 250 and 500 kg/hour.

21. The method of preparing a particulate fat as claimed in claim 20, wherein the product throughput is between 300 and 400 kg/hour.

22. The method of preparing a particulate fat as claimed in claim 11, wherein the pressure across the atomizing nozzle is between 2 and 8 bar.

23. The method of preparing a particulate fat as claimed in claim 22, wherein the pressure across the atomizing nozzle is between 4 and 7 bar.

24. The method of preparing a particulate fat as claimed in claim 23, wherein the pressure across the atomizing nozzle is between 5 and 6 bar.
